(19) United States
(12) Patent Application Publication
(10) Pub. No.: US 2004/0220550 A1
(43) Pub. Date: Nov. 4, 2004

(54) Hybrid extruded articles and method

(76) Inventor: Charles Schryver, Atascadero, CA (US)

Correspondence Address: Michael G. Petit, P. O. Box 91929, Santa Barbara, CA, 93190-1929, US

(21) Appl. No.: 10/778,366

(22) Filed: Feb. 12, 2004

Related U.S. Application Data
(63) Continuation-in-part of application No. 10/154,179, filed on May 24, 2002, now abandoned.

(51) Int. Cl. A61M 27/00

(52) U.S. Cl. 604/544; 428/36.9

(57) ABSTRACT

The present invention discloses extruded articles, such as tubing or multilumen catheters, having hybrid composition, an axial length and a transverse cross-section orthogonal to the length. The extruded articles have unitary construction and require no assembly. The composition of each cross-section of the article along the length of the article is homogeneous and comprises a first extrudable material and/or a second extrudable material. The percentage of the two extrudable materials in any cross-section varies along the length of the article in a controlled, either continuous or discontinuous manner. The first and second extrudable materials are preferably elastomers having different durometers, color or radiopacity. The weight percentage of one of the two extrudable materials in a cross-section may vary abruptly, smoothly and/or periodically along the length of the article.

### BACKGROUND OF THE INVENTION

1. Field of the Invention

The invention relates generally to extruded articles and, more particularly, to extruded elastomeric articles and, even more particularly, to hybrid elastomeric extruded articles.

2. Prior Art

The term “hybrid article”, as used herein, refers to an extruded article having unitary construction and comprised of two or more extrudable materials having different physical or chemical properties. Such hybrid articles are known in the art and are made by means of extrusion through a two chamber extruder as described, for example in U.S. Pat. Nos. 4,282,876 and 5,196,005. A first chamber contains an elastomer having first properties such as, for example, radiopaque silicone or uncured silicone with air bubbles or a foaming agent incorporated therein. The second chamber contains a radiolucent silicone or a degassed silicone elastomer. The elastomer compositions contained with the two separate chambers are simultaneously forced through a single die orifice. The extruded tubing comprises two axially concentric layers of extrudate. The extruder may further include means for varying the shape and size of the die orifice and thus the extruded article's outer diameter as disclosed, for example, in U.S. Pat. No. 5,511,965. In this manner, the extruded hybrid article is formed by extrusion then cured by vulcanization.

U.S. Pat. No. 3,752,617, to Burlis, discloses a method and apparatus for producing tubing having different characteristics along its axial length. In general, '617 provides a method for making tubing either: (a) having a single composition comprising a mixture of extrudable polymers; or (b) having concentric, coaxially disposed inner and outer layers. Burlis also suggests methods for changing the physical properties of the tube as it is extruded. In the first variation, there are two extruders, one for homogenizing and delivering a first polymer to a mixing die and a second extruder for homogenizing and delivering a second polymer to the same mixing die. A sequencing control provides, at various time intervals, an increased flow from one of the extruders and a proportionally decreased flow from the other extruder. The mixture of the two polymers passes through the die and results in an extruded tubing having varying physical parameters along its axial length.

U.S. Pat. No. 4,385,635, to Ruiz, teaches a hybrid angiographic catheter having a soft, flexible, pliable leading tip zone, a main reinforced length, and an intermediate zone between the tip zone and the main length. The main length is made up of a polyamide such as nylon, and the intermediate zone contains a polyamide which is tapered distally and is jacketed by polyurethane. The soft tip is entirely polyurethane. A method for making the device wherein it has unitary construction is not disclosed.

U.S. Pat. No. 4,775,371, to Mueller, Jr., discloses a hybrid balloon catheter having a multilayered shaft. The shaft may be structured in such a fashion that the various layers taper axially in one fashion or another, typically to permit the distal section to be more flexible than the proximal section. However, rather than being coextruded, the various layers are independently extruded. The outer layer comprises a polymer tube which may be fitted over the inner layer and constricted thereonto.

U.S. Pat. No. 5,125,913, to Quackenbush, describes a double-layer medical catheter having an integral soft tip made by the co-extrusion of a relatively rigid inner layer and a relatively soft outer layer. The extrusion equipment is operated to interrupt the supply of the extrudable material comprising the inner, relatively soft layer in order to cause a periodic void in the inner wall as the catheter is extruded. By interrupting the supply of elastomer, a transition in the inner wall thickness occurs at the leading and trailing edge of each void. The void is then cut to produce two soft-tipped catheters.

In accordance with the forgoing teachings, the need for hybrid catheters is well recognized in the art. In certain applications it is desirable to provide an extruded article such as a catheter wherein the composition of the catheter varies along the length of the catheter while the catheter is of unitary construction. For example, it is desirable to provide an indwelling catheter wherein the durometer of the tip or implanted portion of the catheter is different from the extracorporeal portion of the catheter (Martin, U.S. Pat. No. 5,057,073). Until the present invention, such articles either lacked unitary construction, and have been assembled from separate parts, or the limitations of the state of the art prevented or limited the extrusion of custom hybrid catheters having a complex distribution of elastomers along the axial length thereof In another application, it may be desirable to provide a unitary catheter or drain having radiopaque markings thereon, the marks being disposed to indicate a distance along the catheter or, in another application, the radiopaque marks may be employed to indicate, in vivo, the position of a transition between different portions of a drain. The continuing need for such hybrid extrudable articles having unitary construction in versatile, custom configurations and a method for making such articles is met by the present invention.

### SUMMARY

It is an object of the present invention to disclose a hybrid extruded article having unitary construction. The article has a length in the direction of extrusion and a transverse cross-section orthogonal to the length of the article. A cross-sectional portion of the article is comprised of a first weight of a first extrudable material and a second weight of a second extrudable material wherein the ratio of the first weight to the second weight in cross-sectional portions varies along the length of the article.

It is another object of the present invention to disclose a hybrid extruded article having unitary construction wherein the article has a length in the direction of extrusion and a transverse cross-section orthogonal to the length of the article. A cross-sectional portion of the article is comprised of a first extrudable material and a second extrudable material wherein the distribution of the first extrudable material in cross-sectional portions varies along the length of the article.

It is a further object of the invention to provide a method and apparatus operable for making articles meeting the above objectives of the invention.

The features of the invention believed to be novel are set forth with particularity in the appended claims. However the invention itself, both as to organization and method of operation, together with further objects and advantages thereof may be best understood by reference to the following description taken in conjunction with the accompanying drawings in which:

### BRIEF DESCRIPTION OF THE DRAWINGS

FIG. 1 is a plan view of a unitary hybrid article comprising a length of tubing in accordance with a preferred embodiment of the present invention wherein the composition of the tubing varies in a step-wise manner along the length thereof.

FIG. 2a is a front plan view of an extruder die showing the bridge die positioned for the extrusion of a first composition 11 of an extrudable material from the extrusion die.

FIG. 2b is a front plan view of an extruder die showing the bridge die positioned for the extrusion of a second composition 12 of an extrudable material from the extrusion die.

FIG. 3a is a top plan view of an extruder die showing the bridge die positioned for the extrusion of extrudable material 11 from the die.

FIG. 3b is a top plan view of an extruder die showing the bridge die positioned for the extrusion of extrudable material 12 from the die.

FIG. 3c is an enlarged top plan view of the extruder die illustrated in FIG. 3b showing the bridge die positioned for the extrusion of extrudable material 12 from the die.

FIG. 4 is a perspective view of segmented portions of a unitary hybrid article comprising a length of drainage tubing in accordance with a second preferred embodiment of the present invention wherein the distribution and/or amount of a radiopaque extrudable material comprising the drainage tubing varies in a continuous manner along the length thereof.

FIG. 5a is a top plan view of an extruder die operable for the extrusion of a drainage tubing as shown in FIG. 4 showing the flow channel that conducts the radiopaque extrudable material to the die orifice partially obstructed to restrict the flow of radiopaque extrudable material from the extrusion die.

FIG. 5b is a front plan view of an extruder die operable for the extrusion of a drainage tubing as shown in FIG. 4 showing the flow channel that conducts the radiopaque extrudable material to the die orifice partially obstructed to restrict the flow of radiopaque extrudable material into the output orifice of the extrusion die.

FIG. 6a is a top plan view of an extruder die operable for the extrusion of a drainage tubing as shown in FIG. 4 showing the flow channel that conducts the radiopaque extrudable material comprising the stripe to the die orifice partially obstructed to restrict the flow of radiopaque extrudable material from the extrusion die thereby producing a narrow stripe in the extruded drainage tubing.

FIG. 6b is a front plan view of an extruder die operable for the extrusion of a drainage tubing as shown in FIG. 4 showing the flow channel that conducts the radiopaque extrudable material comprising the stripe to the die orifice fully open to maximize the flow of radiopaque extrudable material from the extrusion die therby broadening the stripe in the extruded drainage tubing.

FIG. 7 is a cross-sectional view along section line 7-7 of the tubing of FIG. 1 extruded when the flow channel selector control element comprising the extruder die is positioned as illustrated in FIGS. 2a and 3a.

FIG. 8 is a cross-sectional view along section line 8-8 of the tubing of FIG. 1 formed when the flow channel selector control element comprising the extruder die is positioned as illustrated in FIGS. 2b and 3b.

FIG. 9 is a cross-sectional view along section line 9-9 of the drainage catheter of FIG. 4 extruded when the radiopaque stripe width control element comprising the extruder die is positioned as illustrated in FIGS. 5a and 5b.

FIG. 10 is a cross-sectional view along section line 10-10 of the drainage catheter of FIG. 4, formed when the radiopaque stripe width control element comprising the extruder die is positioned as illustrated in FIGS. 6a and 6b.

FIG. 11 is a graph showing the variation of the weight of a first elastomer (Wgt 1) relative to the total weight of the first elastomer and the weight of a second elastomer (Wgt 1+Wgt 2) in a transverse section of an elongate extruded article in accordance with the present invention sampled at various positions along the length L of the article.

### DESCRIPTION OF THE PREFERRED EMBODIMENTS

The terms: “step-wise” or “step function”, as used herein to indicate the manner in which the percent by weight of a particular elastomer composition in an extruded article comprised of two or more elastomer compositions changes along the length of the article means that the variation in the weight percent of the particular elastomer composition in the article along the length L of the article varies in accordance with FIG. 10.

The term “unitary”, as used herein to describe the construction of an article comprised of two or more elastomer compositions, means that no portions of the article are joined to one another by adhesive means to form a joint between the portions of the article.

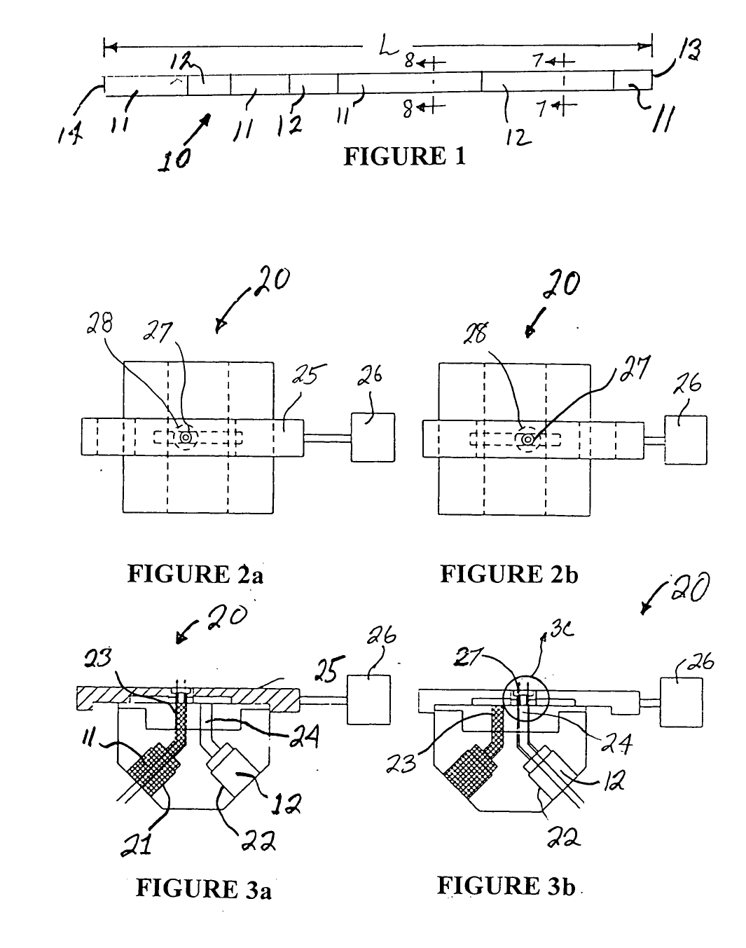

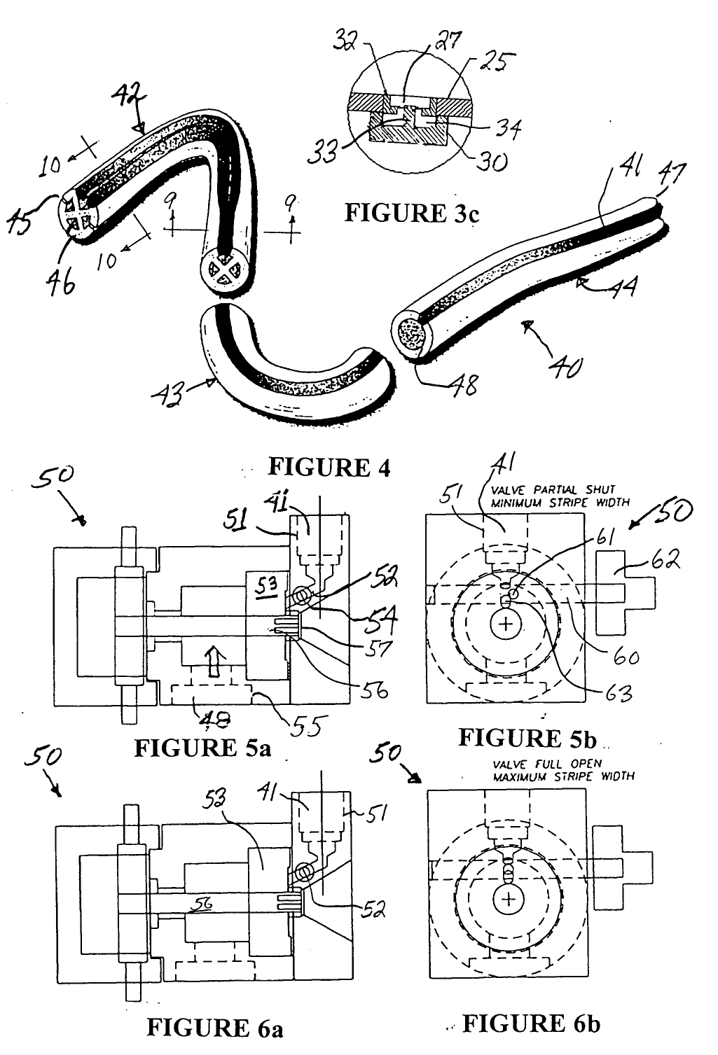

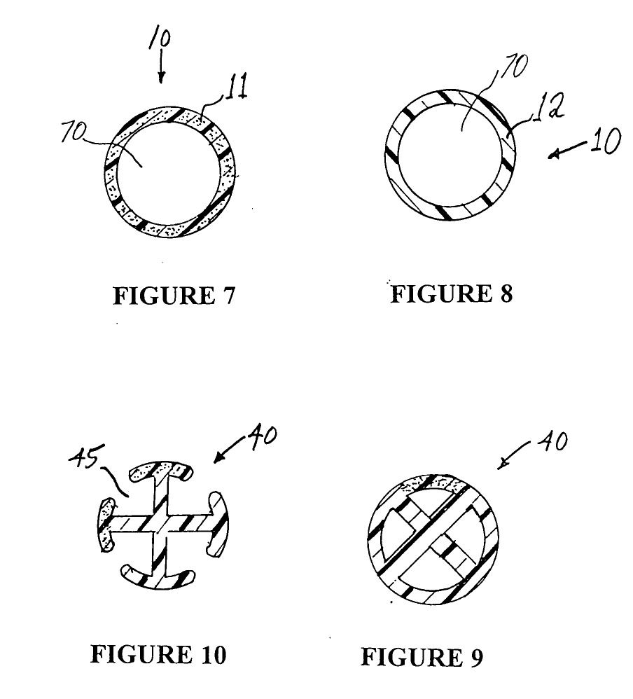

Turning now to FIG. 1, a hybrid extruded tubing in accordance with an embodiment of the present invention is shown in elevational view at numeral 10. The tubing 10 has an axial length L and an axial lumen (not visible in FIG. 1, but indicated at 70 in FIG. 7) coextensive with the length L. The tubing 10 is comprised of alternating amounts of two different elastomeric compositions: a first composition 11 and a second composition 12, coextruded from an extruder die as indicated at numeral 20 in FIGS. 2a-3b. The relative quantities of compositions 11 and 12 within a cross-sectional area of the tubing 10 vary in their distribution along the axial length L of the tubing 10. For example, if the tubing 10 is a catheter with a leading end or tip 13 and a trailing end 14, the composition 11 may be selected to have a durometer, when cured, that is less than the durometer of composition 12 which may form the remaining length of the catheter. In another embodiment, the composition 11 may be radiopaque while the composition 12 is radiolucent. If the percentage of composition 11 varies between 0-100% in discrete incremental distances along the length L, the segments comprised of 100% of composition 11 may be used as markers to determine the position of the tube with respect to tissues within the body.

An extrusion die 20 adapted for making a unitary extruded tubing in accordance with FIG. 1 is shown in FIGS. 2a-3c. Turning first to FIGS. 2a and 3a, a die 20 adapted to extrude the hybrid tubing of FIG. 1 is shown in front view. The die 20 comprises a slidably mounted bridge die plate 25 having an aperture 27 therein. First and second extrudable materials 11 and 12 are contained, under pressure, in reservoirs 21 and 22 having reservoir output nozzles 23 and 24 respectively. The slidably mounted bridge die plate 25 is mechanically or electromagnetically connected to an actuator 26 that controls the position of the aperture 27 with respect to the extrudate reservoir output nozzles 23 and 24. The actuator, which is preferably programmable, forces the aperture 27 to commute reciprocally between the reservoir nozzles 23 and 24 alternately forcing extrudable materials 11 and 12 past the mandrel 33 (FIG. 3c) and through the die aperture 27. FIGS. 2a and 3a illustrate the position of the die aperture 27 when first and second extrudable materials 11 and 12 are respectively forced through the aperture 27.

Surprisingly, even though the commutation of the die aperture 27 between the output nozzles 23 and 24 causes periodic angular displacement of the die aperture 27 with respect to the direction of extrusion of the tubing 10 during the extrusion process, the structural integrity of the extruded tubing is not compromised. Structural irregularity of the extruded tubing due to mechanical displacement of the die aperture during extrusion is minimized by increasing the distance between the die aperture 27 and the vulcanizer (not shown) that receives the extruded tubing 10. A distance of 10-20 inches is adequate. For an extrusion speed of 2-6 in./sec., and a transition (commutation) speed of 12 in./sec., the extrudate would travel 2″/sec X 0.02 sec=0.04″ during the switching operation. Because the flow of either extrudable material 11 or 12 is completely interrupted for part of that time period, the portion of the extruded elastomeric tubing between the die aperture and the downstream vulcanizer probably stretches during the switching time interval, thereby compensating for the interruption in the flow of extrudable material to the die aperture.

With reference now to FIG. 3c, a portion of the bridge die plate 25 is shown in greater detail positioned to receive second extrudable material 12 as indicated in FIG. 3b. The bridge die plate 25 includes a bridge 30 having a mandrel 33 affixed to the bridge 30. A die 32 having an aperture 27 therein is press-fitted into the bridge die plate 25 to complete the assembly of the bridge die plate. The bridge die plate 25 has a receiving chamber 34 that is in material flow communication with nozzle 24 (not shown in FIG. 3c). Second extrudable 12 is forced under pressure into receiving chamber 34 and exits the die aperture 27. After the desired amount of second extrudable material 12 exits the die aperture 27, the actuator repositions the bridge die plate 25 in order to establish material flow communication between the receiving chamber 34 and nozzle 23, permitting the first extrudable material 11 to exit the die aperture 27. Cross-sectional views of tubing 10 taken along section lines 7 and 8 are presented in FIGS. 7 and 8 illustrating the composition of the tubing 10 formed when the bridge die plate is disposed as indicated in FIGS. 3a and 3b respectively.

The “Blake” drain, disclosed in U.S. Pat. No. 4,465,481, has been a mainstay of general surgery since its introduction nearly 20 years ago; providing an efficient, low profile percutaneous drain. The Blake drain normally includes an invasive collection segment having a radiopaque marker integral therewith which enables non-invasive radiographic positioning or re-positioning of the drain segment within the body. A wound drain suitable for insertion into the body and having unitary construction is disclosed by Batdorf et al. in U.S. Pat. No. 5,549,579. The Batdorf drain has a smooth exterior surface and is extruded in a single step to provide a unitary drain having substatially homogeneous elastomeric composition. The Batdorf drain may further comprise a radiopaque marker. Other drains such as, for example, the Jackson-Pratt wound drain are not unitary; comprising a composite structure fabricated by joining separate parts by suitable adhesive means. Such composite drains may disintegrate within the body requiring surgical intervention. While the prior art drains may include a radiopaque marker thereon, it is desirable to provide a drain wherein the radiopaque marker indicates, inter alia, the position of the transition between the collection and the drainage portion of the drain within the body.

FIG. 4 is a perspective view of segmented portions of a unitary hybrid article comprising a length of drainage tubing 40 in accordance with a second preferred embodiment of the present invention wherein the distribution and/or amount of a first extrudable material 41, which is preferably radiopaque, varies in a continuous manner along the length of the drainage tubing. The drain 40 includes a collection segment 42, a transition segment 43 and a extension segment 44. The collection segment 42 is adapted to be inserted within a wound or body cavity thereby providing a fluid-conducting drainage port through which bodily fluids accumulating within a wound or body cavity readily enter and pass for removal from the body. The body fluids (not shown) enter the drain 40 through the lateral grooves 45 which are coextensive with the collection segment; beginning at the distal end of the transition segment 43 and terminating at the distal end 46 of the drain. The body fluids pass through the collection segment 42, into the transition segment 43 and into the extension segment 44 where the fluids are collected by means of gentle suction applied to the proximal end 47 of the drain. The entire drain 40 is a hybrid elastomeric article comprising a radiopaque elastomer 41 and a structural elastomer 48 and having unitary construction. The radiopaque elastomer 41 is present as a single stripe coextensive with the extension portion 44, bifurcating at the distal end of the transition portion 43, the bifurcated stripes of radiopaque elastomer 41 thereafter being coextensive with the collection portion 42 of the drain 40.

The drain 40 is made by extrusion from an extruder die as shown in FIGS. 5a-6b. FIG. 5a is a top plan view of an extruder die 50 operable for the extrusion of a drainage tubing as shown in FIG. 4 showing the flow channel that conducts the radiopaque extrudable material 41 to the extrusion chamber adjacent to the die aperture partially obstructed to restrict the flow of radiopaque extrudable material from the extrusion die. FIG. 5b is a front plan view of the extruder die 50, the structural elements comprising the extrusion die 50 positioned in accordance with FIG. 5a. The radiopaque elastomer 41 is housed within a first pressurized reservoir 51 having a nozzle 52 in material flow communication with an extrusion chamber 53 by means of a valve 54. A second, structural elastomer 48 is housed within a second reservoir 55 and is forced into extrusion chamber 53, under pressure, as indicated by the broad arrow. Since the radiopaque elastomer is injected into the extrusion chamber 53 adjacent to the mandrel 56 and die aperture 57, it is confined to a narrow region around the circumference of the extruded article and forms a stripe thereon.

The operation of the valve 52 is illustrated in FIGS. 5b and 6b. With reference to FIG. 5a, which shows the valve positioned to form a narrow stripe of radiopaque elastomer 41 on the extruded drain 40, the valve 52 comprises a slidably mounted rod 60 having a hole 61 bored transversely therethrough. The rod 60 is in mechanical or electromagnetic connection with an actuator 62. When the rod 60 is retracted in the direction of the actuator, as shown in FIG. 5b, the hole 61 is in misalignment with the lumen 63 of the valve 52, thereby partially obstructing the lumen 63 and restricting the flow of radiopaque elastomer 41 from the reservoir 51.

The mandrel 56 is comprised of two coaxially counted members, only the outermost member being shown in FIGS. 5a-6b. The outermost member is a cylindrical tubing having an axial bore and four prongs on an end thereof adjacent the die aperture 57. A cylindrical rod having a blunt end is slidable mounted therewithin. Both members of the mandrel are connected to actuators such that when the extension portion 44 of the drain 40 is being extruded, the outer, pronged member is retracted and the blunt-ended inner member projects into the die aperture 57. When the transition portion 43 of the drain 40 is formed, the outer, pronged member comprising the mandrel 56 is advanced into the die aperture 57 and the blunt-ended inner rod retracted. Only the pronged end of the outermost member of the mandrel 56 partially occludes the die aperture 57 during extrusion of the collection portion 42 of the drain 40. Cross-sectional views of the extension portion 44 and collection portion 42 of the drain 40 are shown in FIGS. 9 and 10 respectively.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What I claim is:

1. An extruded article having unitary construction, said article having a length and a transverse cross-section orthogonal to said length, said cross-section comprised of a first weight of a first extrudable material and a second weight of a second extrudable material wherein said first and second extrudable materials are homogeneously distributed throughout said transverse cross-section and wherein the ratio of said first weight to said second weight in said transverse cross-section varies along said length of said article.

2. An extruded article in accordance with claim 1 wherein said variation in said ratio of said first weight to said second weight in said cross-section is periodic along said length.

3. The extruded article of claim 2 wherein said variation in said ratio of said first weight to said first weight plus said second weight along said length of said article is substantially a step function.

4. The extruded article of claim 1 wherein said first extrudable material has a first durometer and said second extrudable material has a second durometer that is different than said first durometer.

5. The extruded article of claim 2 wherein said first extrudable material has a first durometer and said second extrudable material has a second durometer that is different than said first durometer.

6. The extruded article of claim 3 wherein said first extrudable material has a first durometer and said second extrudable material has a second durometer that is different than said first durometer.

7. The extruded article of claim 1 wherein said first extrudable material has a radiopacity that is different than the radiopacity of said first material.

8. The extruded article of claim 2 wherein said first extrudable material has a radiopacity that is different than the radiopacity of said first material.

9. The extruded article of claim 3 wherein said first extrudable material has a radiopacity that is different than the radiopacity of said first material.

10. A medical drain operable for collecting and conducting body fluids from the body of a person, the drain having a length and comprising: (a) an extension portion having an axial lumen; (b) a collection portion adapted for implantation within the body, said collection portion thereafter being operable for collecting and conducting body fluids to said axial lumen of said extension portion for conduction from the body; and (c) a radiopaque stripe substantially coextensive with said length of said drain wherein a portion of said radiopaque stripe coextensive with said collection portion is bifurcated wherein said medical drain is of unitary construction.

11. A medical drain in accordance with claim 10 wherein said medical drain is of unitary construction.
